(19) United States
(12) Patent Application Publication
(10) Pub. No.: US 2017/0027677 A1
(43) Pub. Date: Feb. 2, 2017

(54) Dart Delivery System

(71) Applicant: The United States of America, as represented by the Secretary of Agriculture, Washington, DC (US)

(72) Inventors: Jack C Rhyan, Ft. Collins, CO (US); Matthew P. McCollum, Ft. Collins, CO (US)

(21) Appl. No.: 14/809,357

(22) Filed: Jul. 27, 2015

(51) Int. Cl. A61D 7/00; F42B 12/54; A61M 5/20

(52) U.S. Cl. CPC A61D 7/00; A61M 5/20; F42B 12/54; A61M 2005/2013

(57) ABSTRACT

The dart delivery system is designed to be fired from a conventional shotgun. The dart body is enclosed within an outer casing of a shotgun shell. A needle with enclosed medication is connected to the dart body. A fragmentable tip is positioned at the end of the needle. In operation, when the shotgun shell is fired, the dart body and needle are propelled toward a target animal so that when the needle and fragmentable tip contact the skin of the animal, the tip fragments, the needle penetrates the skin, and the powdered, pelleted or lyophilized medication is expelled out the end of the needle and into the animal.

### FIELD OF THE INVENTION

The disclosed method and apparatus relates to a dart delivery system for delivering medication and vaccines. Specifically, the disclosed method and apparatus described herein relates to means for delivering a powdered medication via a shotgun shell-based dart system.

### BACKGROUND OF THE INVENTION

Efficient and humane wildlife management presents multiple challenges—particularly in the context of human encroachment into wildlife habitats. In the shrinking areas that wildlife inhabit, diseases can spread quickly among the wild animals themselves, and they can also spread disease to the human and domestic animal populations that the wildlife come into contact with.

Prior art systems for delivering vaccines, treatment medications, and tranquilizing substances are primarily limited to traditional darts that carry liquid payloads. However, the liquid payloads generally have a short shelf-life. In the case of reconstituted vaccines and certain medications, the vaccines frequently freeze or congeal at colder temperatures. Once a conventional dart is loaded with a liquid vaccine, if not used soon, the dart and its contents must be discarded. Further, traditional darts require an expensive specialized tranquilizer dart gun with a very limited range and a slow, complex, reloading process.

The primary alternative to the traditional dart gun/rifle is the use of a specialized air rifle that fires a “biobullet”. However, the amount and types of medication delivered by a biobullet are very limited. Additionally, accuracy and range are limited and, because biobullets rely on traditional blunt impact to penetrate the skin, the trauma caused by a biobullet is significant.

The need exists for a medication delivery method and apparatus that can accurately deliver a significant quantity of medication at an extended range (up to 100 yards) accurately and with a limited amount of trauma to the targeted animal. The current invention comprises a versatile shotgun shell-based dart delivery vehicle that can deliver a substantial amount of medication accurately through the use of a traditional shotgun so that no specialized “dart rifle” is required.

The system described herein is also less expensive than conventional darting systems. The use of a standard shotgun to deliver darts equates to a decreased initial investment and universally available delivery platforms (i.e. guns). Additionally, the simplicity of design and use of the shotgun shell-based dart systems simplifies the darting process. The shotgun shell-based dart is comprised of inexpensive materials (darts can be produced on a 3D printer) that render the cost of the darts to be significantly less than the cost of conventional darts. Savings will also result from the longer shelf-life of the powdered drugs and vaccines contained in the sealed needles so that the resulting system is (among other things) both more accurate and more efficient than the conventional dart delivery system.

### SUMMARY OF THE INVENTION

This disclosure is directed to a dart delivery system. The system comprises a shotgun shell outer casing designed to enclose a dart body. A needle with enclosed medication is connected to the dart body. A fragmentable tip is positioned at the end of the needle. In operation, when the shotgun shell is fired, the dart body and needle are propelled toward a target animal so that when the fragmentable tip penetrates the skin of the animal, the tip fragments and the medication is expelled out the end of the needle and into the animal.

### BRIEF DESCRIPTION OF THE DRAWINGS

FIG. 1 is a profile view of a dart body and needle along with a traditional shotgun shell in which the dart body is designed to be loaded.

FIG. 2 is a sectional view of a shotgun shell containing a dart body and the associated components of the mechanism described herein.

### DETAILED DESCRIPTION OF PREFERRED EMBODIMENTS

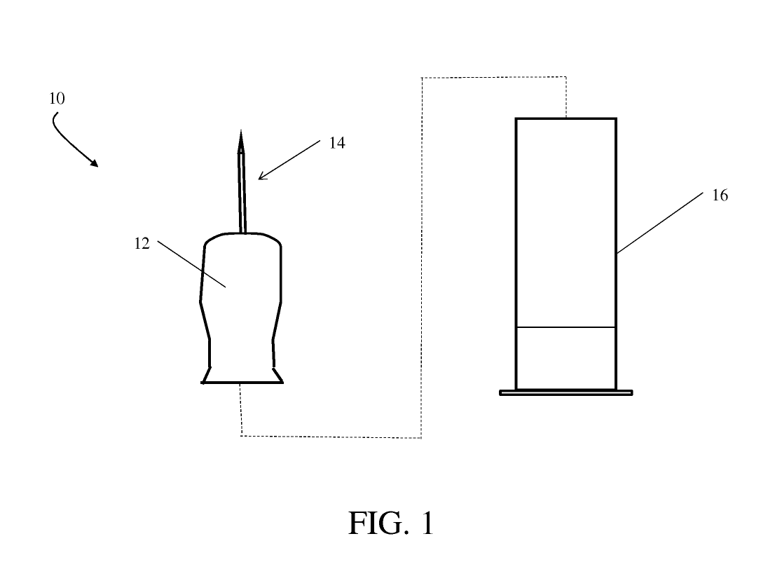

As generally shown in FIG. 1, the apparatus described herein generally comprises a dart-based medication delivery system 10 comprising a dart body 12 and a medication-delivering needle system 14. The shotgun shell-based dart body 12 and needle system 14 are designed to be loaded into a conventional or slightly modified shotgun shell casing 16—so that the loaded shell 16 can be fired from a conventional shotgun and thereby deliver the a dose of medication to a targeted animal.

For the purposes of this disclosure, a “shotgun shell” is a self-contained cartridge that is traditionally loaded with lead shot or a shotgun slug which is designed to be fired from a shotgun. Conventional “shotgun shells” may be 10, 12, 16, 20, or 28 gauge, or they may have a 0.410 inch diameter.

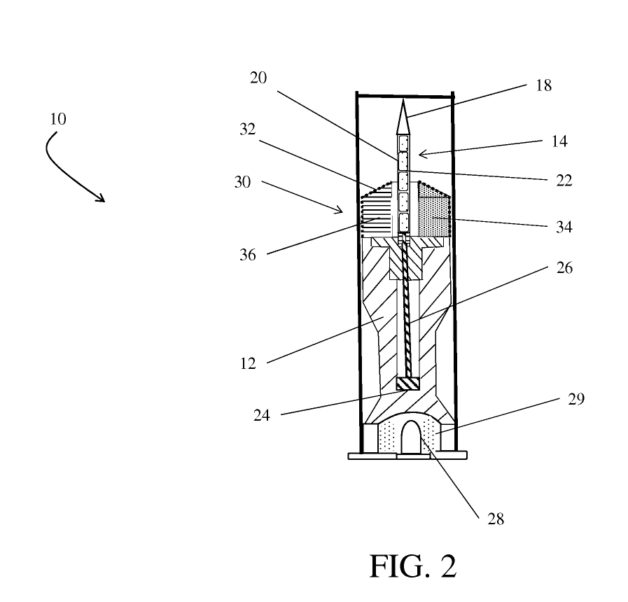

FIG. 2 shows the components of the dart delivery system 10 in greater detail. Specifically, the dart body 12 generally functions as a delivery vehicle for the needle system 14. The needle system 14 comprises a skin piercing tip 18, and a hollow needle body 20 that is designed for delivering a medication 22. A ram rod 26 and associated piston 24 are attached to the base of the needle body so that, in operation, when the needle body 20 impacts a target animal, the piston 24 propels the ramrod 26 forward, which drives the medication 22 out the end of the needle body 20. In the preferred embodiment, the needle body 20 has a luer lock design so that the needle 20 screws onto the dart body 12.

For the purposes of this disclosure, a needle body 20 comprises a hollow cylinder with two oppositely-disposed open ends. Medication 22 is dispersed when pressure at one end of the needle body drives medication out of the oppositely disposed end of the needle.

The dart body 12 is comprised of conventional or biodegradable plastic that is capable of being produced on a 3-D printer. The dart body 12 and associated shell 16 may be designed/sized to be fired from (preferably) a 12 or 20 gauge shotgun. The shotgun shell 16 described herein uses a conventional primer 28 and powder 29 system. The system 10 can also be sized to be shot from a conventional dart rifle with an unmodified 50 caliber barrel.

In the preferred embodiment, the medication 22 delivered by the needle system 14 is powdered, lyophized, or pelletized. The medication 22 is preferably loaded into the needle 20 at a pharmacy or medical facility. The needle 20 is then screwed or otherwise connected to the dart body 12 described herein, or other device incorporating an injection mechanism. In alternate embodiments, the user may load the needle 20 and attach the tip 18 manually. The needle tip 18 is comprised of gelatin or bioplastic and is designed to fragment as the needle tip 18 pierces the skin.

The dart delivery system 10 (optionally) further comprises a marking head 30. As shown in FIG. 2, the marking head 30 is friction fit, screwed, or otherwise attached to the top portion of the dart body 12. The marking head 30 comprises one of at least two types of marking means 34, 36 that are at least partially enclosed in an outer shell 32 comprised of thin plastic, foil, paper, or the like.

As shown in FIG. 2, in accordance with a first embodiment, the marking means 34 comprises a paint, powdered dye, a colored gel, or other type of dispersible colored medium. After the length of the needle shank 20 penetrates the target animal's body, the marking head 30 impacts the animal's skin or otherwise ruptures the marking head's shell 32, causing the colored medium 34 to be dispersed on the outer hair and/or skin of the animal, and thereby marking the area of the needle system's 14 penetration. In the preferred embodiment, the colored medium 34 is designed to contrast with the color of the targeted animal so that a light colored medium would be used with a dark animal (e.g. a buffalo), while a dark color would be used with a light colored animal (e.g. a white-tailed deer).

In accordance with the second embodiment, the marking means 36 comprises a solid marking material such as a helical or non-helical streamer. FIG. 2 shows the streamer 36 coiled/compressed within the marking head 30. An upper portion of the streamer is 36 is saturated with an adhesive so that when the marking head shell 32 ruptures, an end portion of the streamer 36 adheres to the target animal's body in the area that the needle system 14 impacted the animal's body.

In operation, a user selects a needle 20 filled with the desired medication 22 and fitted with a fragmentable tip 18. The user screws or otherwise attaches the needle 20 to a dart body 12 with the ramrod 26 inserted into the back of the needle 20. If the dart body 12 not already enclosed within a conventional shotgun shell-type casing 16, the user loads the assembled dart body 12 and attached needle system 14 into the shell casing 16 to complete the dart-based medication delivery system 10.

When the delivery system 10 is deployed (i.e. the shell casing 16 with the enclosed dart body 12 is loaded into and fired from a shotgun), the dart body 12 and needle system 14 travel accurately up to 100 yards. When the tip 18 of the needle system 14 contacts the skin of a target animal, the tip 18 fragments and the needle 20 penetrates the skin. As the impact with the target animal slows the dart body 12 and needle system 14, inertial force drives the piston 24 and associated ramrod 26 forward so that the medication 22 is injected into the target animal. After the length of the needle body 20 penetrates the target animal's body, the marking head 30 impacts the animal's skin or otherwise ruptures the marking head's shell 32. When the shell 32 ruptures, either a colored medium 34 or a streamer-type marking device 36 within the marking head 30 is deployed to mark the impact point of the needle system 14.

For the foregoing reasons, it is clear that the method and apparatus described herein provides an innovative dart delivery system. The current system may be modified in multiple ways and applied in various technological applications. The disclosed method and apparatus may be modified and customized as required by a specific operation or application, and the individual components may be modified and defined, as required, to achieve the desired result. Although the materials of construction are only generally described, they may include a variety of compositions consistent with the function described herein. Such variations are not to be regarded as a departure from the spirit and scope of this disclosure, and all such modifications as would be obvious to one skilled in the art are intended to be included within the scope of the following claims.

What is claimed is:

1. A dart delivery system comprising:
a shotgun shell outer casing;
a dart body enclosed within the casing;
a needle body connected to the dart body, the needle having a medication enclosed within the needle;
a fragmentable tip positioned at the end of the needle;
wherein, when the shotgun shell is fired, the dart body and the needle are propelled so that when the needle and the fragmentable tip contacts the skin of a target animal, the tip fragments, the needle penetrates the skin, and the medication is expelled out the end of the needle.

2. The system of claim 1 wherein the medication is non-liquid.

3. The system of claim 1 wherein the medication is powdered, lyophized, or pelletized.

4. The system of claim 1 wherein the fragmentable tip is gelatin or bioplastic.

5. The system of claim 1 wherein the needle body has a luer lock type attachment to the dart body.

6. The system of claim 1 wherein the needle screws into the dart body.

7. The system of claim 1 wherein the needle screws or is otherwise attached to the dart body.

8. The system of claim 7 wherein the ramrod is attached to a piston so that when the needle penetrates the target animal, the piston drives the ramrod into the needle so that medication is expelled out an end of the needle.

9. The system of claim 1 further comprising a marking head.

10. The system of claim 9 wherein the marking head is attached to a forward portion of the dart body so that when the dart body impacts the target animal, a colored dye is dispersed on an outer skin of the target animal.

11. The system of claim 8 wherein the marking head is attached to a forward portion of the dart body so that when the dart body impacts the target animal, a marking device adheres to an outer skin of the target animal.

12. The system of claim 11 wherein the marking device is a streamer.

13. A dart delivery system comprising a shotgun shell casing with a needle system and dart body at least partially enclosed within the casing so that, when the shotgun shell is fired, the needle system penetrates skin of a target animal and delivers a medication to the target animal.

14. The system of claim 13 wherein the medication is powdered, lyophized, or pelletized.

15. A method of making a dart delivery system, the method comprising the steps of:
(a) providing a shotgun shell outer casing;
(b) enclosing a dart body within the casing;
(c) connecting a needle body to the dart body, the needle having a medication enclosed within the needle;
(d) positioning a fragmentable tip at the end of the needle.

16. The method of claim 15 wherein the method further comprises step:
(e) connecting a marker head to a top portion of the dart body.

17. The method of claim 16 wherein, in step (e), the marker head comprises one of: a colored medium or a solid streamer.

18. A method of injecting a target animal with a medication, the method comprising the steps of:
(a) providing a shotgun shell outer casing;
(b) enclosing a dart body within the casing;
(c) connecting a needle body to the dart body, the needle having a medication enclosed within the needle;
(d) positioning a fragmentable tip at the end of the needle.
(e) firing the shotgun shell so that when the needle and fragmentable tip contact the skin of a target animal, the tip fragments, the needle penetrates the skin and the medication is expelled out the end of the needle and into the target animal.

19. The method of claim 16 wherein, in step (c), the medication is powdered, lyophized, or pelletized.

20. The method of claim 16 wherein, in step (d), the fragmentable tip is gelatin or bioplastic.
